(19) United States
(12) Patent Application Publication
(10) Pub. No.: US 2015/0141693 A1
(43) Pub. Date: May 21, 2015

(54) PROCESS FOR ACRYLATE PRODUCTION

(71) Applicant: NOVOMER, INC., Ithaca, NY (US)

(72) Inventors: Scott D. Allen, Ithaca, NY (US); Geoffrey Coates, Ithaca, NY (US)

(21) Appl. No.: 14/410,764

(22) PCT Filed: Jul. 2, 2013
(86) PCT No.: PCT/US13/49026

§ 371 Date: Dec. 23, 2014

Related U.S. Application Data
(60) Provisional application No. 61/667,101, filed on Jul. 2, 2012.

(51) Int. Cl. C07C 67/03; C08G 63/08; C07D 305/12

(52) U.S. Cl. CPC C07C 67/03; C07D 305/12; C08G 63/08
USPC 560/204; 560/232

(57) ABSTRACT

Disclosed are methods for the continuous flow production of acrylic acid and derivatives thereof from an epoxide feedstock. In one embodiment, the method includes the steps of: contacting a process stream comprising ethylene oxide and an organic solvent with a carbonylation catalyst and carbon monoxide to provide a reaction stream containing beta propiolactone; applying the reaction stream containing the beta propiolactone to a nanofiltration membrane to produce a permeate stream containing beta lactone and a retentate stream containing carbonylation catalyst; and treating the permeate stream under conditions to convert the beta propiolactone into an acrylate ester. In some embodiments, the retentate stream is returned to the first step of the process where it is recharged with additional epoxide and passed through the sequence again.

### CROSS REFERENCE TO RELATED APPLICATIONS

The present application claims priority to U.S. provisional application Ser. No. 61/667,101 filed Jul. 2, 2012, the entire content of which is hereby incorporated herein by reference.

### FIELD OF THE INVENTION

The invention pertains to the field of chemical synthesis. More particularly, the invention pertains to continuous flow processes for the synthesis of acrylates from epoxide feedstocks.

### SUMMARY OF THE INVENTION

The present invention encompasses methods for the continuous flow production of acrylic acid and derivatives thereof from an epoxide feedstock. In one aspect, shown in Scheme 1, the method includes the steps of: contacting an epoxide 1 with a carbonylation catalyst to yield a beta lactone 2; separating a beta lactone product stream from the carbonylation catalyst; and treating the beta lactone under conditions that cause conversion to an acrylate 3.

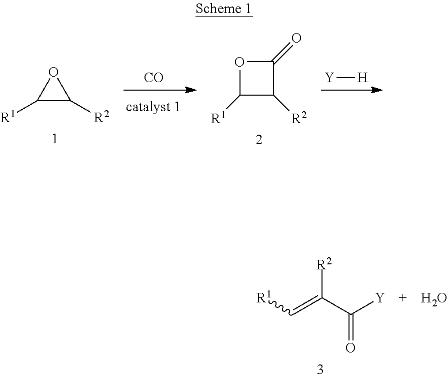

In certain embodiments the carbonylation step is performed in the presence of an organic solvent and the separation of the beta lactone product is performed by nanofiltration on a nanofiltration membrane. This produces two process streams: a permeate stream of the beta lactone product in a portion of the organic solvent passing through the nanofiltration membrane and a retentate stream containing the carbonylation catalyst retained by the nanofiltration membrane and the remainder of the organic solvent. In some embodiments, this retained mixture of organic solvent and carbonylation catalyst is treated as a catalyst recycling stream. In these embodiments, the catalyst recycling stream is returned to the first step of the process where it is recharged with additional epoxide and passed through the sequence again. In some embodiments the permeate stream is distilled to separate the lactone product from the organic solvent. In other embodiments, the permeate stream is fed to an esterification unit prior to the step of treating the beta lactone under conditions that cause conversion to an acrylate (e.g., fed directly to an esterification unit).

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain compounds, as described herein may have one or more double bonds that can exist as either a Z or E isomer, unless otherwise indicated. The invention additionally encompasses the compounds as individual isomers substantially free of other isomers and alternatively, as mixtures of various isomers, e.g., racemic mixtures of enantiomers. In addition to the above-mentioned compounds per se, this invention also encompasses compositions including one or more compounds.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, a compound may, in some embodiments, be provided substantially free of one or more corresponding stereoisomers, and may also be referred to as “stereochemically enriched.” The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation and not aromatic. Unless otherwise specified, aliphatic groups contain 1-30 carbon atoms. In certain embodiments, aliphatic groups contain 1-12 carbon atoms. In certain embodiments, aliphatic groups contain 1-8 carbon atoms. In certain embodiments, aliphatic groups contain 1-6 carbon atoms. In some embodiments, aliphatic groups contain 1-5 carbon atoms; in some embodiments, aliphatic groups contain 1-4 carbon atoms; in yet other embodiments aliphatic groups contain 1-3 carbon atoms; and in yet other embodiments aliphatic groups contain 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroaliphatic”, as used herein, refers to aliphatic groups where one or more carbon atoms are independently replaced by one or more atoms selected from the group consisting of oxygen, sulfur, nitrogen, phosphorus, and boron. In certain embodiments, one or two carbon atoms are independently replaced by one or more of oxygen, sulfur, nitrogen, or phosphorus. Heteroaliphatic groups may be substituted or unsubstituted, branched or unbranched, cyclic or acyclic, and include “heterocycle”, “hetercyclyl”, “heterocycloaliphatic”, or “heterocyclic” groups.

The term “epoxide”, as used herein, refers to a substituted or unsubstituted oxirane.

Substituted oxiranes include monosubstituted oxiranes, disubstituted oxiranes, trisubstituted oxiranes, and tetrasubstituted oxiranes. Such epoxides may be further optionally substituted as defined herein. In certain embodiments, epoxides include a single oxirane moiety. In certain embodiments, epoxides include two or more oxirane moieties.

The term “acrylate” or “acrylates” as used herein refers to any acyl group having a vinyl group adjacent to the acyl carbonyl. The terms encompass mono-, di-, and tri-substituted vinyl groups. Examples of acrylates include, but are not limited to: acrylate, methacrylate, ethacrylate, cinnamate (3-phenylacrylate), crotonate, tiglate, and senecioate. The term “polymer”, as used herein, refers to a molecule of high relative molecular mass, the structure of which includes the multiple repetition of units derived, actually or conceptually, from molecules of low relative molecular mass. In certain embodiments, a polymer includes only one monomer species (e.g., polyethylene oxide). In certain embodiments, a polymer of the present invention is a copolymer, terpolymer, heteropolymer, block copolymer, or tapered heteropolymer of one or more epoxides.

The term “unsaturated”, as used herein, means that a moiety has one or more double or triple bonds.

The term “alkyl”, as used herein, refers to saturated, straight- or branched-chain hydrocarbon radicals derived from an aliphatic moiety containing between one and six carbon atoms by removal of a single hydrogen atom. Unless otherwise specified, alkyl groups contain 1-12 carbon atoms. In certain embodiments, alkyl groups contain 1-8 carbon atoms. In certain embodiments, alkyl groups contain 1-6 carbon atoms. In some embodiments, alkyl groups contain 1-5 carbon atoms, in some embodiments, alkyl groups contain 1-4 carbon atoms, in yet other embodiments alkyl groups contain 1-3 carbon atoms, and in yet other embodiments alkyl groups contain 1-2 carbon atoms. Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like.

The term “alkenyl”, as used herein, denotes a monovalent group derived from a straight- or branched-chain aliphatic moiety having at least one carbon-carbon double bond by the removal of a single hydrogen atom. Unless otherwise specified, alkenyl groups contain 2-12 carbon atoms. In certain embodiments, alkenyl groups contain 2-8 carbon atoms. In certain embodiments, alkenyl groups contain 2-6 carbon atoms. In some embodiments, alkenyl groups contain 2-5 carbon atoms, in some embodiments, alkenyl groups contain 2-4 carbon atoms, in yet other embodiments alkenyl groups contain 2-3 carbon atoms, and in yet other embodiments alkenyl groups contain 2 carbon atoms. Alkenyl groups include, for example, ethenyl, propenyl, butenyl, 1-methyl-2-buten-1-yl, and the like.

The term “alkynyl”, as used herein, refers to a monovalent group derived from a straight- or branched-chain aliphatic moiety having at least one carbon-carbon triple bond by the removal of a single hydrogen atom. Unless otherwise specified, alkynyl groups contain 2-12 carbon atoms. In certain embodiments, alkynyl groups contain 2-8 carbon atoms. In certain embodiments, alkynyl groups contain 2-6 carbon atoms. In some embodiments, alkynyl groups contain 2-5 carbon atoms, in some embodiments, alkynyl groups contain 2-4 carbon atoms, in yet other embodiments alkynyl groups contain 2-3 carbon atoms, and in yet other embodiments alkynyl groups contain 2 carbon atoms. Representative alkynyl groups include, but are not limited to, ethynyl, 2-propynyl (propargyl), 1-propynyl, and the like.

The term “carbocycle” and “carbocyclic ring” as used herein, refers to monocyclic and polycyclic moieties, where the rings contain only carbon atoms. Unless otherwise specified, carbocycles may be saturated, partially unsaturated or aromatic, and contain 3 to 20 carbon atoms. The terms “carbocycle” or “carbocyclic” also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a carbocyclic group is tricyclic. In some embodiments, a carbocyclic group is polycyclic. Representative carbocycles include cyclopropane, cyclobutane, cyclopentane, cyclohexane, bicyclo[2,2,1]heptane, norbornene, phenyl, cyclohexene, naphthalene, and spiro[4.5]decane.

The term “aryl” used alone or as part of a larger moiety as in “aralkyl”, “aralkoxy”, or “aryloxyalkyl”, refers to monocyclic and polycyclic ring systems having a total of five to 20 ring members, where at least one ring in the system is aromatic and where each ring in the system contains three to twelve ring members. The term “aryl” may be used interchangeably with the term “aryl ring”. In certain embodiments of the present invention, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group in which an aromatic ring is fused to one or more additional rings, such as benzofuranyl, indanyl, phthalimidyl, naphthimidyl, phenantriidinyl, tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 14 ring atoms, preferably 5, 6, or 9 ring atoms, having 6, 10, or 14π electrons shared in a cyclic array, and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, where the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or a 7-14-membered bicyclic heterocyclic moiety that is either saturated, partially unsaturated, or aromatic and has, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur, and nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, where the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently a halogen; —(CH₂)₀₋₄R^{∘}; —(CH₂)₀₋₄OR^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)N(R^{∘})₂; —(CH₂)₀₋₄C(O)SR^{∘}; —(CH₂)₀₋₄C(O)OSiR^{∘}₃; —(CH₂)₀₋₄OC(O)R; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, where each R^{∘} may be substituted as defined below and is independently a hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or polycyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently a halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₄C(O)N(R^{∘})₂; —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} where each R^{} is unsubstituted or, where preceded by “halo”, is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, where each independent occurrence of R* is selected from a hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, where each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, where each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; where each R^{†} is independently a hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently a halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, where each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “catalyst” refers to a substance, the presence of which increases the rate of a chemical reaction, while not being consumed or undergoing a permanent chemical change itself.

As used herein, the term “about” preceding one or more numerical values means the numerical value ±5%.

### DETAILED DESCRIPTION OF THE INVENTION

The present disclosure encompasses methods for the production of acrylates from epoxide feedstocks in a continuous-flow process.

In general, processes of the invention include the step of carbonylating an epoxide feedstock to yield a beta lactone-containing process stream. This beta lactone-containing process stream is then transformed to an acrylate product stream by ring opening and dehydration of the lactone.

Turning first to the carbonylation step: in certain embodiments, this step is performed in the presence of an organic solvent by contacting the epoxide with carbon monoxide in the presence of a carbonylation catalyst.

Numerous carbonylation catalysts known in the art are suitable for (or can be adapted to) this step. For example, in certain embodiments, the carbonylation step is performed with a metal carbonyl-Lewis acid catalyst such as those described in U.S. Pat. No. 6,852,865. In other embodiments, the carbonylation step is performed with one or more of the carbonylation catalysts disclosed in U.S. patent application Ser. No. 10/820,958; and Ser. No. 10/586,826. In other embodiments, the carbonylation step is performed with one or more of the catalysts disclosed in U.S. Pat. Nos. 5,310,948; 7,420,064; and 5,359,081. Additional catalysts for the carbonylation of epoxides are discussed in a review in Chem. Commun., 2007, 657-674. The entirety of each of the preceding references is incorporated herein by reference.

In certain embodiments, the carbonylation catalyst includes a metal carbonyl compound. In some embodiments, the metal carbonyl compound has the general formula [QM_{y}(CO)_{w}]^{x}, where:

Q is any ligand and need not be present;

M is a metal atom;

y is an integer from 1 to 6 inclusive;

w is a number selected such as to provide the stable metal carbonyl; and

x is an integer from −3 to +3 inclusive.

In certain embodiments where the metal carbonyl compound has the formula [QM_{y}(CO)_{w}]^{x}, M is selected from the group consisting of Ti, Cr, Mn, Fe, Ru, Co, Rh, Ni, Pd, Cu, Zn, Al, Ga, In and combinations thereof. In certain embodiments, M is Co.

In certain embodiments, the carbonylation catalyst further includes a Lewis acidic component. In some embodiments, the carbonylation catalyst includes an anionic metal carbonyl complex and a cationic Lewis acidic component. In certain embodiments, the metal carbonyl complex includes a carbonyl cobaltate and the Lewis acidic co-catalyst includes a metal-centered cationic Lewis acid.

In certain embodiments, the metal-centered Lewis acid is a metal complex of formula [M′(L)_{b}]^{c+}, where:

M′ is a metal;

each L is a ligand;

b is an integer from 1 to 6 inclusive;

c is 1, 2, or 3; and

where, if more than one L is present, each L may be the same or different.

In some embodiments where the metal-centered Lewis acid is a metal complex of formula [M′(L)_{b}]^{c+}, M′ is selected from the group consisting of: a transition metal, a group 13 or 14 metal, and a lanthanide. In certain embodiments, M′ is a transition metal or a group 13 metal. In certain embodiments, M′ is selected from the group consisting of aluminum, chromium, indium, and gallium. In certain embodiments, M′ is aluminum. In certain embodiments, M′ is chromium.

In certain embodiments, the metal-centered Lewis-acidic component of the carbonylation catalyst includes a dianionic tetradentate ligand. In certain embodiments, the dianionic tetradentate ligand is selected from the group consisting of: a porphyrin derivative; a salen derivative; a dibenzotetramethyltetraaza[14]annulene (tmtaa) derivative; a phthalocyaninate derivative; and a derivative of the Trost ligand.

In certain embodiments, the carbonylation catalyst includes a carbonyl cobaltate in combination with an aluminum porphyrin compound.

In certain embodiments, the carbonylation catalyst includes a carbonyl cobaltate in combination with a chromium porphyrin compound.

In certain embodiments, the carbonylation catalyst includes a carbonyl cobaltate in combination with a chromium salen compound. In certain embodiments, the carbonylation catalyst includes a carbonyl cobaltate in combination with a chromium salophen compound.

In certain embodiments, the carbonylation catalyst includes a carbonyl cobaltate in combination with an aluminum salen compound. In certain embodiments, the carbonylation catalyst includes a carbonyl cobaltate in combination with an aluminum salophen compound.

Solvents suitable for the first step of the process are organic solvents. In certain embodiments, the organic solvent is compatible with the nanofiltration membrane. In certain embodiments, the nanofiltration membrane is stable in the presence of the organic solvent. In certain embodiments, the organic solvent may be chosen from organic solvents including, but not limited to, dimethylformamide, N-methyl pyrrolidone, tetrahydrofuran, toluene, xylene, diethyl ether, methyl-tert-butyl ether, acetone, methylethyl ketone, methyl-iso-butyl ketone, butyl acetate, ethyl acetate, dichloromethane, and hexane, and mixtures of any two or more of these. In general polar aprotic solvents or hydrocarbons are suitable for this step. In certain embodiments, protic solvents are unsuitable for the first step.

In certain embodiments, the catalyst, starting materials, and products are all completely soluble in the organic solvent under the process conditions of the carbonylation step. In other embodiments, one or more of the catalyst, the starting materials, or the products are insoluble or only partially soluble in the organic solvent. In certain embodiments, the carbonylation catalyst is soluble in the organic solvent.

In certain embodiments, one or more additional solvents may be present in the process stream of the first step. In these embodiments, the nanofiltration membrane is stable in the solvent mixture of the process stream, although the nanofiltration membrane may not be stable in one or more of the additional solvents at higher concentrations. In these embodiments, the lactone-containing stream separated in a subsequent step may contain lactone along with one or more of the additional solvents.

In the carbonylation step of the process, there should be enough carbon monoxide present to affect efficient conversion of the epoxide starting material. This can be ensured by performing the reaction under a superatmospheric pressure of carbon monoxide. In certain embodiments, the carbonylation step is performed at a pressure in the range from about 50 psi (350 kPa) to about 5000 psi (35 MPa). In certain embodiments, the carbonylation step is performed at a pressure from about 50 psi (350 kPa) to about 1000 psi (7 MPa). In certain embodiments, the carbonylation step is performed at a pressure from about 50 psi (350 kPa) to about 500 psi (3.5 MPa). In certain embodiments, the carbonylation step is performed at a pressure from about 100 psi (700 kPa) to about 400 psi (2.8 MPa). In certain embodiments, the carbonylation step is performed at a pressure of about 200 psi (1.4 MPa). In certain embodiments, the carbonylation step is performed under an atmosphere having a partial pressure of CO of about 200 psi (1.4 MPa).

The superatmospheric pressure of carbon monoxide may be provided in the form of pure carbon monoxide, or by providing a gas mixture containing carbon monoxide. In certain embodiments, the carbon monoxide may be provided in the form of substantially pure carbon monoxide. In other embodiments, the carbon monoxide may be provided in the form of carbon monoxide mixed with one or more inert gases. In other embodiments, the carbon monoxide may be provided in the form of a mixture of carbon monoxide and hydrogen. In certain embodiments, the carbon monoxide may be provided in the form of a carbon monoxide-containing industrial process gas such as syngas, coal gas, wood gas, or the like.

The temperature of the first step should be maintained in a range where the catalyst, the starting materials, and the products of the carbonylation reaction are stable for the duration of the process, and at a temperature at which the carbonylation reaction proceeds at a rate that allows conversion of starting material in a convenient and economical time-frame. In certain embodiments, the step is performed at a temperature in the range of about −10° C. to about 200° C. In certain embodiments, the step is performed at a temperature in the range of about 0° C. to about 125° C. In certain embodiments, the step is performed at a temperature in the range of about 30° C. to about 100° C. In certain embodiments, the step is performed at a temperature in the range of about 40° C. to about 80° C.

In certain embodiments, the epoxide starting material has the formula

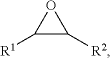

where R¹ and R² are each independently selected from the group consisting of: —H; optionally substituted C₁₋₆ aliphatic; optionally substituted C₁₋₆ heteroaliphatic; optionally substituted 3- to 6-membered carbocycle; and optionally substituted 3- to 6-membered heterocycle, where R¹ and R² can optionally be taken together with intervening atoms to form a substituted or unsubstituted ring optionally containing one or more heteroatoms.

In certain embodiments, the epoxide is chosen from the group consisting of: ethylene oxide; propylene oxide; 1,2-butylene oxide; 2,3-butylene oxide; epichlorohydrin; cyclohexene oxide; cyclopentene oxide; 3,3,3-Trifluoro-1,2-epoxypropane, styrene oxide; a glycidyl ether; and a glycidyl ester.

In certain embodiments, the epoxide is ethylene oxide.

In certain embodiments, the epoxide is propylene oxide.

In certain embodiments, step 1 includes the reaction shown in Scheme 2:

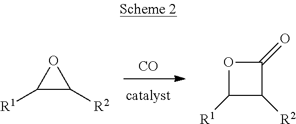

where R¹ and R² are each independently selected from the group consisting of: —H; optionally substituted C₁₋₆ aliphatic; optionally substituted C₁₋₆ heteroaliphatic; optionally substituted 3- to 6-membered carbocycle; and optionally substituted 3- to 6-membered heterocycle, where R¹ and R² can optionally be taken together with intervening atoms to form a substituted or unsubstituted ring optionally containing one or more heteroatoms.

In certain embodiments, step 1 includes the reaction shown in Scheme 3:

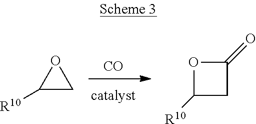

where, R¹⁰ is selected from the group consisting of —H, and C₁₋₆ aliphatic.

In certain embodiments, step 1 includes the reaction shown in Scheme 4:

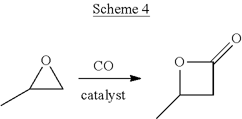

In certain embodiments, step 1 includes the reaction shown in Scheme 5:

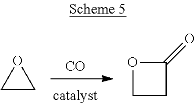

In certain embodiments, the first step is conducted in a continuous flow process whereby the starting epoxide is continuously fed into a reaction stream and the carbonylation takes place as the reaction stream flows through the process. In some embodiments, the epoxide fed into the process is substantially consumed and the reaction stream flowing out of the process contains little or no residual epoxide starting material. It will be understood by those skilled in the art that the process parameters such as reaction temperature, carbon monoxide pressure, catalyst loading, epoxide concentration, agitation, path length, and flow rate, can all be optimized to affect this end.

In certain embodiments, the carbonylation step is performed in a process stream flowing through an adiabatic reaction vessel. In certain embodiments, the adiabatic reaction vessel is a tube reactor. In other embodiments, the carbonylation step is performed in a process stream flowing through a shell and tube reactor.

A subsequent step in processes of the present invention separates the carbonylation catalyst from the propiolactone in the process stream resulting from the carbonylation step described above. This step produces two new process streams: a lactone stream containing the lactone and a catalyst recycling stream.

In some embodiments, this separation is performed by exposing the lactone-containing process stream to a nanofiltration membrane. The nanofiltration membrane is preferably an organic solvent-stable nanofiltration membrane. Although any nanofiltration membrane may be used in combination with any organic solvent or organic solvent system compatible with the carbonylation reaction and the nanofiltration membrane within the spirit of the present invention, the nanofiltration membrane is preferably selected in combination with the organic solvent or solvents such that the process achieves predetermined levels of lactone formation and catalyst-lactone separation. In some embodiments, the nanofiltration membrane is chosen from nanofiltration membranes including, but not limited to, polyimides, including those marketed under the trademark STARMEM by Membrane Extraction Technology Ltd (Wembley, UK) and integrally skinned asymmetric membranes made from polyimides, polyamide-imides, silicone-coated polyamide composites, polyacrylonitriles, polydimethylsiloxane films on polyacrylonitrile supports, silcones, polyphosphazenes, polyphenylene sulfide, polyetheretherketone, and polybenzimidazol. In some embodiments, the organic solvent is tetrahydrofuran and the nanofiltration membrane is an integrally skinned asymmetric polyimide membrane made from Lenzing P84 or a STARMEM® polyimide membrane. In some embodiments, the organic solvent is diethyl ether and the nanomembrane is a silicone-coated polyamide composite.

In some embodiments, the nanofiltration membrane is a commercially available membrane. In other embodiments, the nanofiltration membrane is an integrally skinned asymmetric polyimide membrane made from Lenzing P84 and manufactured by GMT Membrantechnik GmbH (Rheinfelden, Germany). In some other embodiments, the nanofiltration membrane is a STARMEM® polyimide membrane from Membrane Extraction Technology Ltd (Wembley, UK) and the nanofiltration step is performed at a temperature under 50° C. and a pressure under 60 bar. In still other embodiments, the nanofiltration membrane is a silicone-coated organic solvent resistant polyamide composite nanofiltration membrane as disclosed in U.S. Pat. No. 6,887,380, incorporated herein by reference.

The permeate stream resulting from the nanofiltration step is carried onto an acrylate production step. The acrylate production step is discussed in more detail below. The permeate stream may optionally be processed in a number of ways prior to the acrylate production step. This processing can include, but is not limited to: vacuum-distilling, heating, cooling, or compressing the stream; condensing the stream to a liquid state and carrying forward the liquid; adding a polymerization inhibitor to the stream; condensing selected components to a liquid state and carrying forward the remaining gaseous components; condensing selected components to a liquid state and carrying forward the liquefied components; scrubbing the stream to remove impurities; and any combination of two or more of these.

The other stream resulting from the nanofiltration step is the retentate stream or catalyst recycling stream. In certain embodiments, this stream is returned to the beginning of the process where it re-enters the carbonylation step and is brought into contact with additional epoxide and carbon monoxide. In certain embodiments, the catalyst recycling stream is treated prior to re-entering the carbonylation process. Such treatments can include, but are not limited to: filtering, concentrating, diluting, heating, cooling, or degassing the stream; removing spent catalyst; removing reaction byproducts; adding fresh catalyst; adding one or more catalyst components; and any combination of two or more of these.

Turning next to the acrylate production step, the permeate stream discussed above is carried onward to convert the beta lactone contained therein to acrylic acid or an acrylic acid derivative. As discussed above, in some embodiments, the permeate stream may undergo additional processing steps between the nanofiltration step and the acrylate production step and may enter the acrylate production stage of the process as a gas or as a liquid. The acrylate production step itself may be performed in either the gas phase or the liquid phase and may be performed either neat, or in the presence of a carrier gas, solvent or other diluent.

In certain embodiments, the acrylate production step is performed in a continuous flow format. In certain embodiments, the acrylate production step is performed in a continuous flow format in the gas phase. In certain embodiments, the acrylate production step is performed in a continuous flow format in the liquid phase. In certain embodiments, the acrylate production step is performed in a liquid phase in a batch or semi-batch format.

The acrylate production step may be performed under a variety of conditions. In certain embodiments, the reaction may be performed in the presence of one or more catalysts that facilitate one or more steps in the transformation of the beta lactone intermediate to the acrylate product. Many catalysts known in the art can be used, or adapted for this step. In some embodiments, conditions include reaction with dehydrating agents such as sulfuric acid, phosphoric acid or esters thereof as described in U.S. Pat. Nos. 2,352,641; 2,376,704; 2,449,995; 2,510,423; 2,623,067; 3,176,042, and in British Patent No. GB 994,091, the entirety of each of which is incorporated herein by reference.

In other embodiments, the lactone can be reacted with a halogenic compound to yield a beta halo acid, beta halo ester, or beta halo acid halide which may then undergo dehydrohalogenation and/or solvolysis to afford the corresponding acrylic acid or acrylic ester. In certain embodiments, conditions disclosed in U.S. Pat. No. 2,422,728 (incorporated herein by reference) are used in this process.

In other embodiments, the acrylate production may be base catalyzed, see for example Journal of Organic Chemistry, 57(1), 389-91(1992) and references therein, the entirety of which is incorporated herein by reference.

In certain embodiments, the acrylate production stage of the process may be performed by combining the permeate stream from the previously described steps with an alcohol vapor and passing the mixture in the gas phase through a column of a solid, or solid supported promoter that effects the conversion to an acrylic ester. In certain embodiments, this process is performed over a promoter including activated carbon according to the methods of U.S. Pat. No. 2,466,501 the entirety of which is incorporated herein by reference.

In some embodiments, the beta lactone in the permeate stream is allowed to polymerize and acrylic acid or derivatives thereof are obtained by decomposition of the polymer. In certain embodiments, the beta lactone is propiolactone and the polymer is poly(3-hydroxy propionic acid) (3-HPA). In certain embodiments, the 3-HPA is formed and decomposed using the methods described in U.S. Pat. Nos. 2,361,036; 2,499,988; 2,499,990; 2,526,554; 2,568,635; 2,568,636; 2,623,070; and 3,002,017, the entirety of each of which is incorporated herein by reference.

In certain embodiments, the beta lactone product stream is reacted with a nucleophile of the formula Y—H. In certain embodiments, Y is selected from the group consisting of halogen; —OR¹³; —NR¹¹R¹²; and —SR¹³, where R¹¹, R¹², and R¹³ are independently selected from the group consisting of: —H; optionally substituted C₁₋₃₂ aliphatic; optionally substituted C₁₋₃₂ heteroaliphatic; optionally substituted 3- to 14-membered carbocycle; and optionally substituted 3- to 14-membered heterocycle, and where R¹¹ and R¹² can optionally be taken together with intervening atoms to form an optionally substituted ring optionally containing one or more heteroatoms.

In certain embodiments, the beta lactone product stream is reacted with a nucleophile of the formula Y—H to afford an acrylate having the formula I:

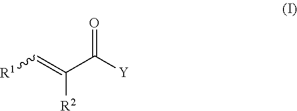

In certain embodiments, Y—H is an amine having the formula R¹¹R¹²N—H, and the product is an acrylamide. In certain embodiments, this process uses conditions disclosed in U.S. Pat. Nos. 2,548,155; 2,649,438; 2,749,355; and 3,671,305, the entirety of each of which is incorporated herein by reference.

In certain embodiments, the beta lactone product stream is reacted with a nucleophile of the formula Y—H to afford an acid having the formula II:

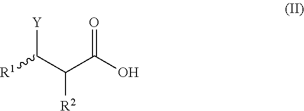

In certain embodiments, compounds of formula II are obtained using conditions disclosed in U.S. Pat. Nos. 2,449,992; 2,449,989; 2,449,991; 2,449,992; and 2,449,993, the entirety of each of which is incorporated herein by reference.

In certain embodiments, where the beta lactone product stream is reacted with a nucleophile of the formula Y—H to afford an acid having the formula II, and Y is —OR¹³; —NR¹¹R¹²; or —SR¹³, the acid is dehydrated to yield an acrylate of formula I.

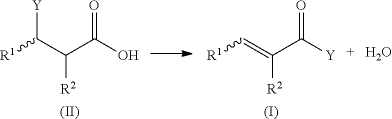

In certain embodiments, the conversion of II to I is performed according to the methods and conditions of U.S. Pat. No. 2,376,704 the entirety of which is incorporated herein by reference.

In certain embodiments, the acrylate product stream resulting from the preceding steps may undergo additional purification steps. In certain embodiments, the stream is purified according to methods disclosed in U.S. Pat. Nos. 3,124,609; 3,157,693; 3,932,500; 4,828,652; 6,084,122; 6,084,128; and 6,207,022, the entirety of each of which is incorporated herein by reference.

In certain embodiments, the present invention includes methods for the production of acrylates from epoxides in a continuous flow process, the process including the steps of a) contacting a process stream including an epoxide and an organic solvent with a carbonylation catalyst in the presence of carbon monoxide to provide a reaction stream containing a beta lactone formed from the epoxide, where the organic solvent is compatible with a nanofiltration membrane, b) applying the reaction stream to a nanofiltration membrane to produce a carbonylation product stream including beta lactone and a first portion of the organic solvent and a catalyst recycling stream including carbonylation catalyst and a second portion of the organic solvent, and c) treating the carbonylation product stream under conditions to convert the beta lactone into an acrylate.

In certain embodiments, the process further includes the step of returning the catalyst recycling stream to step a).

In certain embodiments, the process further includes treating the catalyst recycling stream by performing at least one step selected from the group consisting of adding fresh catalyst, removing spent catalyst, adding solvent, adding epoxide, and any combination of two or more of these.

In some embodiments, step c) of the process is performed in the presence of a compound selected from the group consisting of: an alcohol, an amine, and a thiol, under conditions that afford the corresponding acrylic ester, acrylamide, or a thioacrylate respectively.

In certain embodiments, the invention provides a method for the production of an acrylate ester from ethylene oxide in a continuous flow process, the method comprising the steps of:

a) contacting a process stream comprising ethylene oxide and an organic solvent with a carbonylation catalyst in the presence of carbon monoxide to provide a reaction stream containing beta propiolactone formed from the ethylene oxide;

b) applying the reaction stream containing the beta propiolactone to a nanofiltration membrane to produce:
i) a permeate stream comprising beta propiolactone and a first portion of the organic solvent, and
ii) a retentate stream comprising carbonylation catalyst and a second portion of the organic solvent; and

c) treating the permeate stream under conditions to convert the beta propiolactone into an acrylate ester;

optionally further comprising the step of returning the retentate stream to step (a);
optionally further comprising treating the retentate stream prior to returning it to step (a) where the step of treating is selected from the group consisting of: adding fresh catalyst, removing spent catalyst; adding solvent; adding epoxide; and any combination of two or more of these.

In certain embodiments, the invention provides a method for the production of poly(3-hydroxy propionic acid) from ethylene oxide in a continuous flow process, the method comprising the steps of:

a) contacting a process stream comprising ethylene oxide and an organic solvent with a carbonylation catalyst in the presence of carbon monoxide to provide a reaction stream containing beta propiolactone formed from the ethylene oxide;

b) applying the reaction stream containing the beta propiolactone to a nanofiltration membrane to produce:
i) a permeate stream comprising beta propiolactone and a first portion of the organic solvent, and
ii) a retentate stream comprising carbonylation catalyst and a second portion of the organic solvent; and

c) treating the permeate stream under conditions to convert the beta propiolactone into poly(3-hydroxy propionic acid);

optionally further comprising the step of returning the retentate stream to step (a);

optionally further comprising treating the retentate stream prior to returning it to step (a) where the step of treating is selected from the group consisting of: adding fresh catalyst, removing spent catalyst; adding solvent; adding epoxide; and any combination of two or more of these. It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method for the production of an acrylate ester from ethylene oxide in a continuous flow process, the method comprising the steps of:
a) contacting a process stream comprising ethylene oxide and an organic solvent with a carbonylation catalyst in the presence of carbon monoxide to provide a reaction stream containing beta propiolactone formed from the ethylene oxide;
b) applying the reaction stream containing the beta propiolactone to a nanofiltration membrane to produce:
i) a permeate stream comprising beta propiolactone and a first portion of the organic solvent, and
ii) a retentate stream comprising carbonylation catalyst and a second portion of the organic solvent; and
c) treating the permeate stream under conditions to convert the beta propiolactone into an acrylate ester.

2. A method for the production of poly(3-hydroxy propionic acid) from ethylene oxide in a continuous flow process, the method comprising the steps of:
a) contacting a process stream comprising ethylene oxide and an organic solvent with a carbonylation catalyst in the presence of carbon monoxide to provide a reaction stream containing beta propiolactone formed from the ethylene oxide;
b) applying the reaction stream containing the beta propiolactone to a nanofiltration membrane to produce:
i) a permeate stream comprising beta propiolactone and a first portion of the organic solvent, and
ii) a retentate stream comprising carbonylation catalyst and a second portion of the organic solvent; and
c) treating the permeate stream under conditions to convert the beta propiolactone into poly(3-hydroxy propionic acid).

3. The method of claim 1, further comprising the step of returning the retentate stream to step (a).

4. The method of claim 3, further comprising treating the retentate stream prior to returning it to step (a) where the step of treating is selected from the group consisting of: adding fresh catalyst, removing spent catalyst; adding solvent; adding epoxide; and any combination of two or more of these.

5. The method of claim 1, wherein the nanofiltration membrane is selected from the group consisting of a polyimide membrane, an integrally skinned asymmetric polyimide membrane, a polyamide-imide membrane, a silicone-coated polyamide composite membrane, a polyacrylonitrile membrane, a membrane comprising a polydimethylsiloxane film on a polyacrylonitrile support, a silicone membrane, a polyphosphazene membrane, a polyphenylene sulfide membrane, a polyetheretherketone membrane, a polybenzimidazol membrane, and combinations thereof.

6. The method of claim 1, wherein the carbonylation catalyst comprises a metal carbonyl compound.

7. The method of claim 6, wherein the metal carbonyl compound has the general formula [QM_{y}(CO)_{w}]^{x},
where: Q is any ligand and need not be present;
M is a metal atom;
y is an integer from 1 to 6 inclusive;
w is a number such as to provide the stable metal carbonyl; and
x is an integer from −3 to +3 inclusive.

8. The method of claim 7, wherein M is selected from the group consisting of Ti, Cr, Mn, Fe, Ru, Co, Rh, Ni, Pd, Cu, Zn, Al, Ga, In and combinations thereof; or where M is Rh; or where M is Co.

9. The method of claim 6, wherein the carbonylation catalyst further comprises a Lewis acidic co-catalyst.

10. The method of claim 9, wherein the metal carbonyl compound is anionic, and the Lewis acidic co-catalyst is cationic.

11. The method of claim 10, wherein the metal carbonyl compound comprises a carbonyl cobaltate and the Lewis acidic co-catalyst comprises a metal-centered Lewis acid.

12. The method of claim 11, wherein the metal-centered Lewis acid is a metal complex of formula [M′(L)_{b}]^{c+},
where, M′ is a metal;
each L is a ligand;
b is an integer from 1 to 6 inclusive;
c is 1, 2, or 3; and
where, if more than one L is present, each L may be the same or different.

13. The method of claim 12, where M′ is selected from the group consisting of aluminum, chromium, indium and gallium; or where M′ is aluminum; or where M′ is chromium.

14. The method of claim 12, where the metal-centered Lewis acid includes a dianionic tetradentate ligand; or where the metal-centered Lewis acid includes a dianionic tetradentate ligand selected from the group consisting of: a porphyrin derivative; a salen derivative; a dibenzotetramethyltetraaza[14]annulene (tmtaa) derivative; a phthalocyaninate derivative; and a derivative of the Trost ligand; or where the metal-centered Lewis acid includes a porphyrin ligand.

15. The method of claim 1, wherein the permeate stream is fed to an esterification unit prior to step (c).

16. The method of claim 1 further comprising the step of vacuum distilling the permeate stream to separate the beta lactone from the first portion of the organic solvent prior to step (c).

17. The method of claim 1, wherein step (c) is mediated by a catalyst.

18. The method of claim 17, wherein the catalyst in step (c) is an acid catalyst; or wherein the catalyst in step (c) is a basic catalyst.

19. The method of claim 1, wherein step (a) is performed at a CO pressure from about 50 psi to about 5000 psi.

20. The method of claim 1, wherein step (a) is performed at a temperature from about 0° C. to about 125° C.; or
wherein step (a) is performed at a temperature from about 30° C. to about 100° C.; or
wherein step (a) is performed at a temperature from about 40° C. to about 80° C.
